(19) United States
(12) Patent Application Publication
(10) Pub. No.: US 2022/0241554 A1
(43) Pub. Date: Aug. 4, 2022

(54) IMPLANTABLE CATHETER

(71) Applicant: W. L. Gore & Associates, Inc., Newark, DE (US)

(72) Inventors: Jérôme S. Conia, Phoenix, AZ (US); Edward H. Cully, Newark, DE (US); Jared S. Nelson, Flagstaff, AZ (US); Cedomila Ristic-Lehmann, Flagstaff, AZ (US)

(21) Appl. No.: 17/629,680

(22) PCT Filed: Jul. 24, 2020
(86) PCT No.: PCT/US2020/043365

§ 371 Date: Jan. 24, 2022

Related U.S. Application Data
(60) Provisional application No. 62/878,130, filed on Jul. 24, 2019.

(51) Int. Cl. A61M 25/00; A61M 5/158

(52) U.S. Cl. CPC A61M 25/0017; A61M 5/158; A61M 2210/1017; A61M 25/0075; A61M 25/0074

(57) ABSTRACT

Various aspects of the present disclosure are directed toward apparatuses, systems, and methods that are configured to be implanted within a patient. The apparatuses, systems, and methods may include a catheter configured to implant within the intraperitoneal space of the patient, an interior flow lumen, and at least one opening connected to the interior flow lumen for therapeutic agent delivery.

### CROSS-REFERENCE TO RELATED APPLICATION

This application is a national phase application of PCT Application No. PCT/US2020/043365, internationally filed on Jul. 24, 2020, which claims the benefit of Provisional Application No. 62/878,130, filed Jul. 24, 2020, each of which are incorporated herein by reference in their entireties for all purposes.

### FIELD

The present disclosure relates generally to catheters, and more specifically to apparatuses, systems, and methods that include catheters that may be implanted into a patient.

### BACKGROUND

Catheters or other similar devices for long term implantation may have issues with long term patency. For example, depending on the location site of implantation, catheters or other similar devices may induce a physiological response such as foreign body reaction or inflammation. This response can lessen the ability of the implanted catheters or other similar devices to function as desired.

### SUMMARY

According to one example (“Example 1”), an apparatus configured to be implanted within a patient includes a catheter including a proximal section and a distal section configured to implant within the patient, an interior flow lumen, and at least one opening connected to the interior flow lumen for therapeutic agent delivery; and a cover arranged about at least a portion of the distal section and configured to lessen at least one of a foreign body response, inflammation, and cellular ingress and maintain the opening substantially unobstructed for drug delivery through the catheter.

According to another example (“Example 2”), further to the apparatus of Example 1, the cover is an ePTFE film.

According to another example (“Example 3”), further to the apparatus of any one of Examples 1-2, the cover extends along the portion of the distal section between about 1 mm to about 100 mm from a distal end of the catheter.

According to another example (“Example 4”), further to the apparatus of any one of Examples 1-3, the catheter is configured for drug delivery to the intraperitoneal space through the interior flow lumen and the cover includes a drug distribution material.

According to another example (“Example 5”), further to the apparatus of Example 4, the catheter is an indwelling catheter configured to implant within the intraperitoneal space for up to 20 years.

According to another example (“Example 6”), further to the apparatus of any one of Examples 1-5, at least one opening is arranged at the distal end of the catheter.

According to another example (“Example 7”), further to the apparatus of any one of Examples 1-6, the at least one opening includes a plurality of openings spaced about a circumference of the distal section of the catheter to enable uniform distribution of the therapeutic agent.

According to another example (“Example 8”), further to the apparatus of Example 7, the cover is arranged over the plurality of openings.

According to another example (“Example 9”), further to the apparatus of any one of Examples 1-8, the apparatus also includes a sealed tip arranged at the distal end of the catheter.

According to another example (“Example 10”), further to the apparatus of any one of Examples 1-9, the apparatus also includes an interior layer arranged within the catheter along the interior flow lumen configured to lessen a foreign body response and inflammation.

According to another example (“Example 11”), further to the apparatus of any one of Examples 1-10, the apparatus also includes at least one of a bioactive agent or bioactive cover arranged on an exterior surface of the catheter.

According to another example (“Example 12”), further to the apparatus of any one of Examples 1-11, the apparatus also includes a self-closing tube section arranged at a distal end of the elongate body.

According to another example (“Example 13”), further to the apparatus of Example 12, the self-closing tube section is configured to open in response to pressure from a pump that forces the therapeutic agent through the elongate body and close in response to the absence of the pressure.

According to another example (“Example 14”), further to the apparatus of any one of Examples 1-11, the apparatus also includes a catheter tip section arranged at a distal end of the elongate body that includes a valve configured to open in response to pressure from a pump that forces the therapeutic agent through the elongate body and close in response to the absence of the pressure.

According to another example (“Example 15”), further to the apparatus of any one of Examples 1-11, the apparatus also includes a pressure distended elastomeric tip arranged at a distal end of the elongate body that includes an opening configured to open in response to pressure from a pump that forces the therapeutic agent through the elongate body and close in response to the absence of the pressure.

According to one example (“Example 16”), a method of treatment includes providing a catheter including a proximal section, a distal section, an interior flow lumen, and at least one opening connected to the interior flow lumen for therapeutic agent arranged at a distal end of the catheter; inserting the distal end of the catheter into a patient; and introducing the therapeutic agent to the interior flow lumen so that the therapeutic agent is delivered into the patient through at least one opening.

According to another example (“Example 17”), further to the method of Example 16, the catheter also includes a cover arranged about at least a portion of the distal section and configured to lessen at least one of a foreign body response, inflammation, and cellular ingress and maintain the opening substantially unobstructed for delivery of the therapeutic agent through the catheter.

According to another example (“Example 18”), further to the method of Example 17, the cover is comprised of an ePTFE film.

According to another example (“Example 19”), further to the method of Examples 17-18, the cover extends along the portion of the distal section between about 1 mm to about 100 mm from the distal end of the catheter.

According to another example (“Example 20”), further to the method of Examples 16-19, the at least one opening includes a plurality of openings spaced about a circumference of the distal section of the catheter to enable uniform distribution of the therapeutic agent.

According to another example (“Example 21”), further to the method of Examples 16-20, the method also includes the step of controlling a flow of the therapeutic agent delivery with a pump.

According to another example (“Example 22”), further to the method of Examples 16-21, the therapeutic agent comprises insulin.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is an example catheter system arranged within a patient, in accordance with various aspects of the present disclosure.

FIG. 2 is an example catheter for implantation within a patient, in accordance with various aspects of the present disclosure.

FIG. 3 is another example catheter for implantation within a patient, in accordance with various aspects of the present disclosure.

FIG. 4 is another example catheter for implantation within a patient, in accordance with various aspects of the present disclosure.

FIG. 5 is another example catheter for implantation within a patient, in accordance with various aspects of the present disclosure.

FIG. 6 is another example catheter for implantation within a patient, in accordance with various aspects of the present disclosure.

FIG. 7 is another example catheter for implantation within a patient, in accordance with various aspects of the present disclosure.

FIG. 8 is an example catheter tip, in accordance with various aspects of the present disclosure.

FIG. 9 is another example catheter tip, in accordance with various aspects of the present disclosure.

FIG. 10 is yet another example catheter tip, in accordance with various aspects of the present disclosure.

FIG. 11A is an example catheter tip in a first configuration, in accordance with various aspects of the present disclosure.

FIG. 11B is the catheter tip, shown in FIG. 11A, in a second configuration, in accordance with various aspects of the present disclosure.

FIG. 12 is another example catheter tip, in accordance with various aspects of the present disclosure.

FIG. 13 is another example catheter tip, in accordance with various aspects of the present disclosure.

### DETAILED DESCRIPTION

### Definitions and Terminology

As the terms are used herein with respect to ranges of measurements “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

This disclosure is not meant to be read in a restrictive manner. For example, the terminology used in the application should be read broadly in the context of the meaning those in the field would attribute such terminology.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example. In the event it is determined that individuals having ordinary skill in the relevant arts would not readily ascertain values for such reasonably small differences, the terms “about” and “approximately” can be understood to mean plus or minus 10% of the stated value.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying figures referred to herein are not necessarily drawn to scale but may be exaggerated to illustrate various aspects of the present disclosure, and in that regard, the drawing figures should not be construed as limiting. It is to be noted that the terms “catheter system” and “system” may be used interchangeably herein.

Various aspects of the present disclosure are directed to apparatus, systems, and methods that include a catheter configured to implant within a patient. The catheters may include one or more exterior layers that lessen a physiological response that occurs when a foreign body or device is implanted within the intraperitoneal space. In certain instances, the physiological response may lessen the ability of the catheter to function as intended. The catheters, as discussed in further detail below, lessen the physiological response in order to maintain functionality of the catheter.

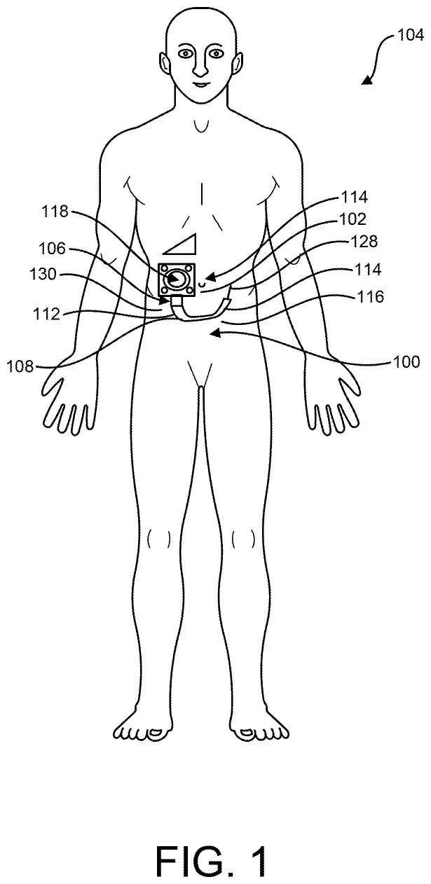

FIG. 1 is an example of a catheter system 100 arranged within an intraperitoneal space 102 of a patient 104, in accordance with various aspects of the present disclosure. The intraperitoneal space 102 is located between muscles and organs in the abdomen and includes a peritoneal lining with bodily fluid present between the peritoneal lining and the organs. In certain instances, it may be preferred to use intraperitoneal therapy rather than intravenous injection when large amounts of blood replacement fluids are needed or when low blood pressure or other clinical and/or procedural complications prevent the use of a suitable blood vessel for such intravenous injection. Intraperitoneal therapy may also be preferred over subcutaneous therapy as a more direct physiologic method and therefore provide a potentially superior response to the therapy.

A system 100 used for intraperitoneal therapy may include an access port 106 and a catheter 108 in fluid communication with the access port 106. The access port 106 may be placed into a pocket formed under the skin within subcutaneous tissue of a body of the patient 104 (e.g., within the lower or upper abdomen) and the catheter extends from the access port 106 into the peritoneal space 102. In an illustrative embodiment, the catheter 108 is a thin, flexible tube comprised of silicone or another compliant polymer.

During intraperitoneal therapy, a reagent, medication, fluid products, nutrients, and/or another therapeutic agent may be mixed with fluids and injected directly into the peritoneal space 102 through the access port 106 and catheter 108. In some embodiments, a pump 118 may be utilized with the catheter system 100, which includes a reservoir that maintains a supply of the therapeutic agent (e.g., reagent or liquid) to the catheter 108 on a controlled basis. A monitor may also be used in conjunction with the catheter system 100. In other embodiments, the catheter 108 may be placed in other spaces of the body (e.g. lower back) to direct treatment.

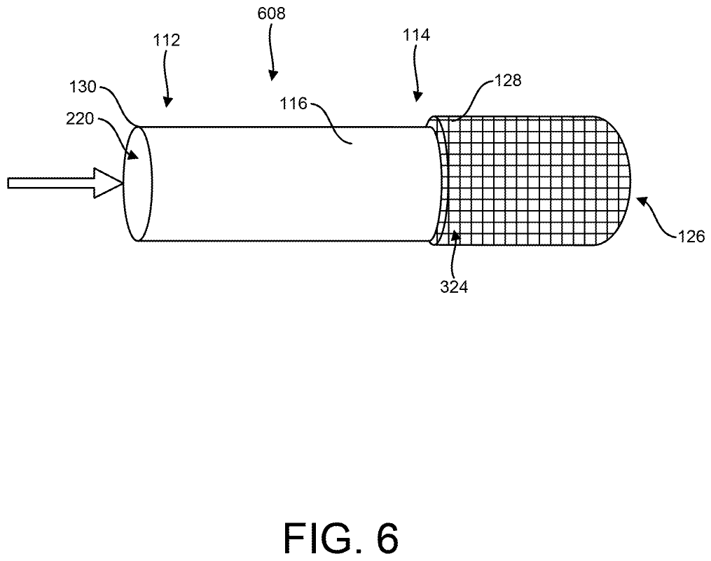

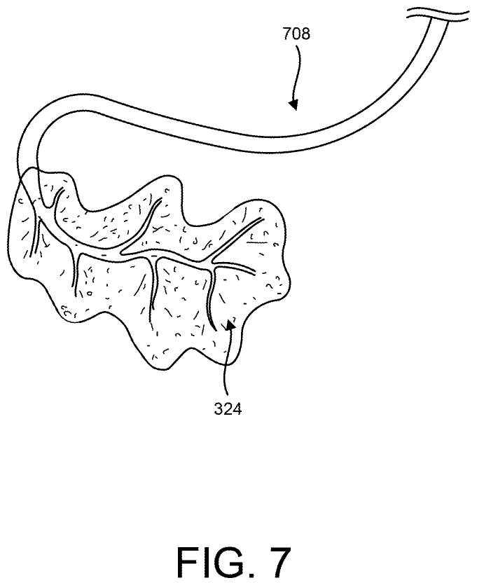

In certain instances, the pump 118 may be implanted in the subcutaneous territory (e.g., underneath the skin). The pump 118 may be configured to release the medical or therapeutic agent into the intraperitoneal cavity (e.g., at the biological interface with the distribution material as described with reference to FIG. 6 and FIG. 7).

As illustrated in FIG. 1, the catheter 108 includes a proximal section 112 near the access port 106 and a distal section 114. At least the distal section 114 is configured to implant within the intraperitoneal space 102 of the patient 104. The catheter 108 includes an elongate body 116 that generally forms a cylindrical shape, although other shapes may be utilized and are considered within the purview of the invention. For example, the catheter 108 may be formed into a shape representing any number of different polygons or other shapes such as those utilizing a curved portion. The elongate body 116 of the catheter 108 forms an interior flow lumen which enables fluid contact with at least one opening in the elongate body 116 coupled to the interior flow lumen for delivery of medication, fluid products, nutrients, and/or another therapeutic agent. In certain instances, the catheter 108 may be used for collection or sampling of fluid.

For example, the elongate body 116 of the catheter 108 may also define an opening at either a proximal end 130 of the proximal section 112 or a distal end 128 of the distal section 114 of the catheter 108 that is directly connected to the interior flow lumen of the catheter 108 to provide access to the interior flow lumen and the intraperitoneal space 102 for delivery of the therapeutic agent that may include a medication or other therapeutic agent. In certain instances, the distal end 128 may be the portion of the catheter 108 exposed to the intraperitoneal space. In an illustrative embodiment, a cover (abluminal and/or luminal) may be arranged about at least a portion of the distal section 114 of the catheter 108 to lessen a foreign body response or inflammation that may occur as a result of the insertion of the catheter 108 into the body of the patient 104, as further discussed below. Such a cover may additionally maintain an opening at the distal end 128 of the catheter 108 substantially unobstructed to facilitate the delivery of the therapeutic agent that may include a medication or other therapeutic agent through the catheter 108. The lumen of the catheter 108 may also be coated with an active component such as heparin, which may lessen foreign body response. The catheter 108 may be soft (e.g., tissue compliant) and flexible to maintain conformability and comfort for the patient 104 without kinking and/or to minimize tissue irritation.

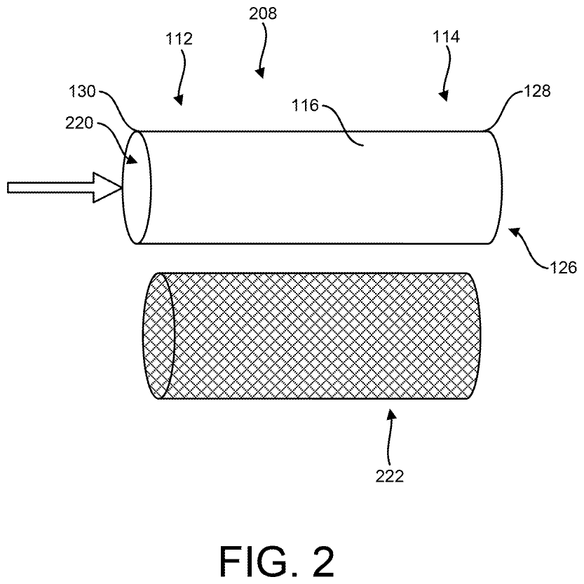

FIG. 2 is an example catheter 208 for implantation within a patient, in accordance with various aspects of the present disclosure. In certain instances, the catheter 208 includes a silicone elongate body 116 which defines an inner flow lumen 220. In other embodiments, the elongate body 116 of the catheter 208 may include other polymers such as polyurethane. A wall of the inner flow lumen 220 may include or be coated with a polymer layer. In certain instances, the polymer layer may prevent a pH change of the drug or other therapeutic agent that is to be delivered. For example, the wall of inner flow lumen 220 of the catheter 208 may include a layer of or otherwise be coated with polyethylene, which serves as a barrier to permeation of carbon dioxide from the environment around the catheter, to maintain (or otherwise not influence) the pH balance of insulin delivered to the intraperitoneal space of a patient with diabetes. In other instances, other polymers or hydrophilic materials may be used corresponding to the therapeutic agent being used in treatment. The elongate body 116 and the wall of the inner flow lumen 220 (and/or outer surface) may further be coated with heparin, dexamethasone, or another bioactive agent to minimize fibrotic cell encapsulation inside or around the catheter 208.

In certain instances, the catheter 208 may also include a cover 222, which is configured to cover substantially the entirety of the elongate body 116 of the catheter 208. The cover 222 may include a fluoropolymer such as polytetrafluoroethylene (PTFE) or expanded polytetrafluoroethylene (ePTFE). In some instances, the inner flow lumen 220 may also include a layer of or be coated with a fluoropolymer such as PTFE or ePTFE. The cover 222 may be bound to the catheter 208 through an adhesive, such as liquid silicone rubber or another polymer adhesive that is biocompatible. Liquid silicone rubber or another adhesive may be applied to the cover 222 or the elongate body 116 of the catheter 208 and the sleeve then wrapped around the elongate body 116 of the catheter 208 so that the adhesive forms a bond between the cover 222 and the elongate body 116.

The catheter 208 includes a proximal section 112 which may be coupled to an access port 106 as shown in FIG. 1 and a distal section 114 which may be implanted within an intraperitoneal space 102 of a patient 104. The distal section 114 of the catheter 208 is shown in FIG. 2. The catheter 208 (or a distal section of the catheter 208) may be configured to remain in the intraperitoneal space 102 of the patient 104 for any period of time including years and up to 20 years (e.g., barring infection or non-performance). In other instances, the catheter 208 may remain in the intraperitoneal space of the patient for a shorter or longer period of time. The elongate body 116 of the catheter 208 may form a cylindrical shape, although other shapes may be utilized. The elongate body 116 forms the inner flow lumen 220 which enables fluid contact with at least one opening connected to the inner flow lumen 220 for delivery of medication, fluid products, nutrients, or other therapeutic agent(s).

For example, the elongate body 116 of the catheter 208 may also include an opening 126 at a distal end 128 of the distal section 114 of the catheter 208 that is directly connected to the interior flow lumen 220 of the catheter 208 to provide an exit point for the interior flow lumen 220 into the intraperitoneal space for delivery of a therapeutic agent that may include a medication or other therapeutic agent(s).

The cover 222 arranged about an outer surface of the elongate body 116 of the catheter 208 may be configured to lessen a foreign body response or inflammation that may occur as a result of the implantation of the catheter 208 within the body of the patient. The cover 222 may additionally maintain a substantially unobstructed opening 126 at the distal end 128 of the catheter 208 to facilitate the delivery of a liquid that may include a medication or therapeutic agent through the inner flow lumen 220 of the catheter 208. In certain instances, the catheter 208 may include an additional sleeve arranged within the interior flow lumen 220 of the catheter 208. The sleeve may further lessen a foreign body response, lessen further fibrotic encapsulation, or lessen inflammation within the catheter 208. Additionally, the elongate body 116 of the catheter 208 may be coated with a bioactive agent to further discourage fibrotic cell encapsulation, flow obstruction of a therapeutic agent, or other undesirable effects of the catheter insertion into the intraperitoneal space of the patient.

In certain instances, the microstructure of the cover 222 is configured to lessen the opportunity for fibrotic encapsulation. In certain instances, an open porous microstructure of the cover 222 is designed to allow and/or encourage cell ingrowth and/or lessen the opportunity for fibrotic encapsulation. In other instances, an ePTFE structure having small nodes and short fibrils (a tight structure) may be used as a porous microstructure to prevent fibrotic encapsulation. In certain instances, the cover 222 enables continuous outflow of the therapeutic agent that may include a medication or another therapeutic agent and uptake of the therapeutic agent by the tissue surrounding the catheter 208. In other instances, the cover 222 may include another biocompatible material (additionally or in alternative to) configured to inhibit or otherwise discourage fibrotic encapsulation while enabling continuous outflow from the catheter.

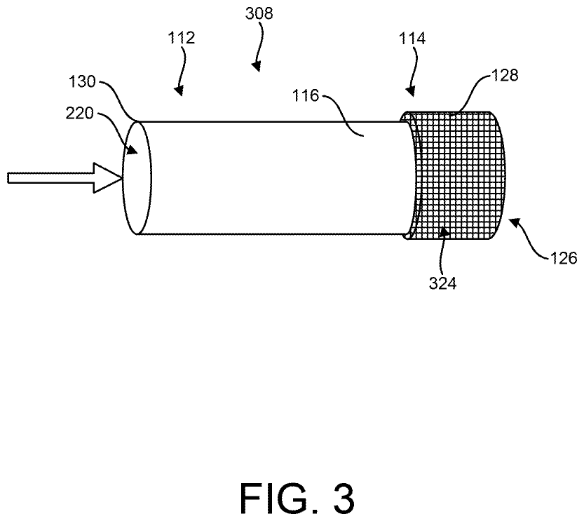

FIG. 3 is another example catheter 308 for implantation within a patient, in accordance with various aspects of the present disclosure. The catheter 308 may share many of the characteristics of the catheters 108, 208 discussed above, including an elongate body 116, an inner flow lumen 220, a proximal section 112, and a distal section 114.

The catheter 308 may further comprise a distal cover 324 that covers a portion of the distal section 114 of the catheter 308 adjacent to the opening 126. In certain instances, the distal cover 324 includes a fluoropolymer such as polytetrafluoroethylene (PTFE) or expanded polytetrafluoroethylene (ePTFE). In addition, the distal cover 324 may include an ePTFE membrane cap that inhibits cellular infiltration and minimizes inflammation/fibrotic encapsulation while enabling continuous therapeutic agent outflow. In certain instances, the distal cover 324 may include an ePTFE membrane cap with an outer surface layer with microporous structure engineered to minimize foreign body response and an inner surface layer with microstructure configured to serve as a filtration membrane and prevent ingress of cells into the lumen of the catheter 308 and allow transport of a therapeutic agent out of the catheter 308 into the surrounding tissue.

The cover 324 may be bound to the catheter 308 through an adhesive, such as liquid silicone rubber or another polymer adhesive that is biocompatible. In certain instances, liquid silicone rubber or another adhesive may be applied to the cover 324 or the elongate body 116 of the catheter 308 and the sleeve then placed into contact with the elongate body 116 at the point of bonding so that the adhesive forms a bond between the cover 324 and the elongate body 116 while allowing the cover 324 to maintain an unobstructed opening 126 of a distal end 128 of the distal section 114 of the catheter 308. The cover 324 may extend along the distal section 114 from about 1 mm to about 100 mm from the distal end 128 of the catheter 308. The cover 324 may extend an entire length of the catheter 308 in certain instances. In certain instances, the cover 324 may extend along the distal section 114 of approximately 5%-25% of the length of the catheter 308.

The proximal section 112 of catheter 308 may be coupled to an access port 106 as shown in FIG. 1 and the distal section 114 of catheter 308 may be implanted within an intraperitoneal space 102 of a patient 104. In certain instances, the catheter 308 may remain in the intraperitoneal space 102 of the patient 104 for any period of time spanning the life of the catheter. The elongate body 116 has an inner flow lumen 220 which enables fluid contact or communication with at least one opening connected to the interior flow lumen 220 for delivery of medication, fluid products, nutrients, or liquids.

The cover 324, arranged about at least a portion of the distal section 114 of the catheter 308, is configured to lessen a foreign body response or inflammation that may occur as a result of the implantation of the catheter 308 into the body of the patient. In certain instances, the cover 324 may extend along the distal section 114 of the catheter 308 and wrap around the distal end 226 and into the interior flow lumen 220 to cover or coat a portion of the interior flow lumen 220. An additional cover 324 may be coupled to or arranged within the catheter 308 along the interior flow lumen 220. In certain instances, this additional cover 324 may further lessen a foreign body response or inflammation (e.g., resulting from the insertion of the catheter 308 into the body of the patient 104). Additionally, the elongate body 116 of the catheter 308 may be coated with a bioactive agent to discourage fibrotic cell encapsulation or other unfavorable effects of the catheter insertion.

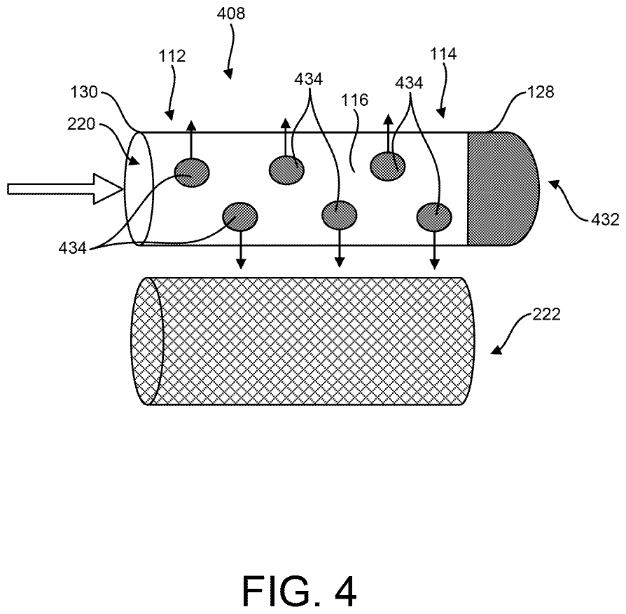

FIG. 4 is another example catheter 408 for implantation within a patient, in accordance with various aspects of the present disclosure. As shown in FIG. 4, a distal end 128 of the catheter 408 may include a sealed tip 432. The sealed tip 432 may be spherical, flat, rounded, or polygonal shaped. The sealed tip 432 closes off the catheter 408, thus, the catheter 408 includes openings 434 along the elongate body 116 and in contact with the inner flow lumen 220 to enable the dispersion of the therapeutic agent. In certain instances, the openings 434 are spaced within the distal section 114 of the catheter 408 to enable a therapeutic agent (e.g., reagent or liquid) to disperse in the intraperitoneal space. In certain instances, the plurality of openings 434 are spaced about a circumference of the distal section 114 of the catheter 408 to enable uniform distribution of the therapeutic agent. In other instances, the openings 434 may be present substantially along the entirety of the catheter 408.

As shown in FIG. 4, the openings 434 may be drilled, formed, punctured, or otherwise made through the elongate body 116 in a radial arrangement. Other arrangements of openings 434 may be utilized, and other methods of creating the openings 434 in the elongate body 416 may be utilized. The openings 434 are not limited to use in the catheter 408 shown in FIG. 4 but may also be used in conjunction with catheters 108 shown in FIGS. 1-3. Similarly, catheters 108 shown in FIGS. 1-3 may also include a sealed tip similar to sealed tip 432 shown in FIG. 4.

The catheter 408 also includes a cover 222, which covers at least the openings 434 located in the distal section 114 of the catheter 408. In an illustrative embodiment, the cover 222 includes a semi-permeable material (e.g., ePTFE) that inhibits fibrotic cellular infiltration while enabling continuous therapeutic agent outflow. In certain instances, the microstructure of the cover 222 is controlled to inhibit cellular infiltration while remaining semi-permeable to various therapeutic agents. For example, the cover 222, having an ePTFE structure, may include nodes with fibrils to provide a semi-permeable microstructure in which the pores are large enough to allow drug molecule flow, insulin dispersion, or dispersion of other therapeutic agents and result in a microstructure that is open enough to allow such dispersion. The cover 222 may enable continuous outflow of the therapeutic agent and uptake of said therapeutic agent by the tissue surrounding the catheter 408. In other instances, the cover 222 may enable growth of vascular neovessels into its porous structure, thus reducing the distance between delivery and uptake of a therapeutic agent and enabling minimum lag time. In other instances, the cover 222 may provide a structure that is both a barrier to cell ingress while maintaining unhindered therapeutic outflow. In other instances, the cover 222 may be comprised of another biocompatible material configured to inhibit or otherwise discourage cellular infiltration and fibrotic encapsulation while enabling continuous outflow.

As described in detail above, the catheter 408 may be coupled to an access port 106 as shown in FIG. 1 and the distal section 114 of catheter 408 may be implanted within an intraperitoneal space 102 of a patient 104.

The catheter 408 shown in FIG. 4 is provided as an example of the various features of the cover 222 and, although the combination of those illustrated features is clearly within the scope of invention, that example and its illustration is not meant to suggest the inventive concepts provided herein are limited from fewer features, additional features, or alternative features to one or more of those features shown in FIG. 4. For example, in various embodiments, the catheter 408 shown in FIG. 4 may include the distal cover 324 described with reference to FIG. 3. It should also be understood that the reverse is true as well. One or more of the components depicted in FIG. 4 can be employed in addition to, or as an alternative to components depicted in the other figures. For example, the covers 222 discussed herein may include the microstructure properties discussed in detail above.

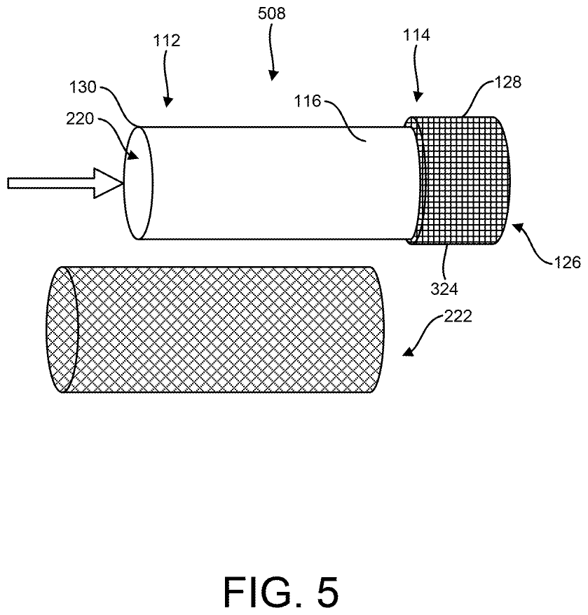

FIG. 5 is another example catheter 508 for implantation within a patient, in accordance with various aspects of the present disclosure. The catheter 508 may share many of the characteristics of the catheter 408 discussed above, including an elongate body 116, an inner flow lumen 220, a proximal section 112, and a distal section 114.

The catheter 508 also includes a cover 222. In an illustrative embodiment, the cover 222 (e.g., as described in FIG. 4 or FIG. 5) includes a semi-permeable material (e.g., ePTFE) that inhibits fibrotic cellular infiltration while enabling continuous therapeutic agent outflow. In certain instances, the microstructure of the cover 222 is controlled to inhibit fibrotic cellular infiltration while remaining semi-permeable to various therapeutic agents. For example, the cover 222, having an ePTFE structure, may include nodes with fibrils to provide a semi-permeable microstructure (e.g., permeable to certain size particles or elements and non-permeable to other size particles or elements) in which the pores are large enough to allow drug molecule volume flow, insulin dispersion, or dispersion of other therapeutic agents and result in a microstructure that is open enough to allow such dispersion. The cover 222 may enable continuous outflow of the therapeutic agent and uptake of said therapeutic agent by the tissue surrounding the catheter 508. In other instances, the cover 222 may be comprised of another biocompatible material configured to inhibit or otherwise discourage fibrotic encapsulation while enabling continuous outflow through the distal cover 324.

The catheter 508 may further include a distal cover 324 that covers a portion of the distal section 114 of the catheter 508 adjacent the opening 126. In certain instances, the distal cover 324 includes a fluoropolymer such as PTFE or ePTFE. In addition, the distal cover 324 may include an ePTFE membrane cap that inhibits fibrotic cellular infiltration and minimizes inflammation/fibrotic encapsulation while enabling continuous therapeutic agent outflow. For example, the distal cover 324, having an ePTFE structure, may include nodes with fibrils to provide a semi-permeable microstructure (e.g., permeable to certain size particles or elements and non-permeable to other size particles or elements) in which the pores are large enough to allow drug molecule volume flow, insulin dispersion, or dispersion of other therapeutic agents and result in a microstructure that is open enough to allow such dispersion while also small enough pores that would prevent cellular ingress. The distal cover 324 may enable continuous outflow of the therapeutic agent and uptake of said therapeutic agent by the tissue surrounding the catheter 508.

To lessen a foreign body response, inflammation and maintain the opening substantially unobstructed for drug delivery through the catheter 508, the distal cover 324 and/or elongate body cover 222 may be formed of ePTFE, the permeability of which and ability to encourage mesothelial cell ingrowth is determined by the pore size and material thickness. The pore size is measured at the material surface by determining the internodal distance or fibril length of the material. Fibril length may be measured as described in U.S. Pat. No. 4,482,516. The ePTFE fibril length in a single or in more than one direction is estimated as the average of several measurements between nodes connected by fibrils in the various directional orientations of stretching. The cover 324 and cover 222 may have different permeabilities.

The fibril length and the thickness of ePTFE materials are chosen to either resist or accept cellular ingrowth across a fraction or across the entire length and/or thickness of the elongate body 116 of the catheter 508 (e.g., corresponding to the location of the cover 324 and/or cover 222). The structure of the elongate body 116 of the catheter 508 may be a laminate with variable permeability (e.g., different sections of permeability), such as a cell permeable layer adjoining the exterior surface of the catheter and a cell exclusion layer adjacent to the interior flow lumen 220. The cell permeable and cell exclusion layers of the catheter 508 each may contribute to the overall thickness of the catheter wall construct in either equal or asymmetrical proportions, the thickness of each material ranging from 1 microns to about 2,500 microns.

The cell permeable layer may have an average pore size greater than about 3.0 microns, and in certain instances, the pore size may be greater than about 5.0 microns. The cell exclusion layer is impermeable to cellular ingrowth, preventing cells from entering the interior flow lumen 220, and contacting, adhering to, fouling, ingrowing, overgrowing, or otherwise interfering with the therapeutic agent or drug delivered through the catheter 508. To exclude invading host cells, the average pore size of the exclusion layer may range from less than about 3.0 microns to 0.1 micron.

The catheter 508 shown in FIG. 5 is provided as an example of the various features of the cover 324 and/or cover 222, although the combination of those illustrated features is clearly within the scope of invention, that example and its illustration is not meant to suggest the inventive concepts provided herein are limited from fewer features, additional features, or alternative features to one or more of those features shown in FIG. 5. For example, in various embodiments, the catheter 508 shown in FIG. 5 may include the radial openings 434 described with reference to FIG. 4. It should also be understood that the reverse is true as well. One or more of the components depicted in FIG. 5 can be employed in addition to, or as an alternative to components depicted in the other figures. For example, the covers 222 discussed herein may include the microstructure properties discussed in detail above.

FIG. 6 is another example catheter 608 for implantation within a patient, in accordance with various aspects of the present disclosure. The catheter 608 may share many of the characteristics of the catheter 508 discussed above, including an elongate body 116, an inner flow lumen 220, a proximal section 112, and a distal section 114. The elongate body 116 of the catheter 108 may also define an opening at either a proximal end 130 of the proximal section 112 or a distal end 128 of the distal section 114 of the catheter 108 that is directly connected to the interior flow lumen of the catheter 108 to provide access to the interior flow lumen and the intraperitoneal space 102 for delivery of the therapeutic agent that may include a medication or other therapeutic agent.

The catheter 608 may further comprise a distal cover 324 that covers a portion of the distal section 114 of the catheter 608 adjacent to the opening 126. In certain instances, the distal cover 324 is configured to facilitate distribution of the therapeutic agent or drug delivered through the catheter 608. Similar to FIG. 6, FIG. 7 is another example catheter 708 for implantation within a patient, in accordance with various aspects of the present disclosure, that includes the distal cover 324 configured to facilitate distribution of the therapeutic agent or drug delivered through the catheter 708. The distal cover 324 of FIG. 7 includes is a drug distribution leaflet or includes a drug distribution leaflet. The distal cover 324 including distribution properties may be kink resistant and may be collapsible to eliminate dead volume. The leaflet may be made of drug distribution material for the therapeutic administration across a wide range of blood and lymph capillaries. The catheter 608 and catheter 708 may be coupled to the pump 118 described in detail above to drive the therapeutic agent or drug for delivery.

In certain instances, the cover 324 being configured as a drug distribution material may disperse the therapeutic agent or drug by wicking or other dispersion methods over a wide biological area. This dispersion method may facilitate access to a large number of blood or lymph capillaries within the host tissues and enables natural pharmacokinetics, ensuing a more benign healing response.

The cover 324 being configured as a drug distribution material may integrate microstructures, including fibrillated polymeric materials that exhibit selective permeability (e.g.: fluoropolymer, thin ePTFE membranes, composite films and bio-absorbable substrates), to facilitate establishing an interface between the therapeutic agent or drug and surrounding body fluids, dissolved gases, or gases that could otherwise alter the properties of the drug. In certain instances, the cover 324 may include a fibrillated polymeric diffusion material configured to exhibit permeability to macromolecules of a molecular weight consistent with targeted clinical applications. In certain instances, the fibrillated fabric may be configured by its thickness, pore size, fibril length, and the orientation of the assorted fibrils. Fluid transport through the fibrillated polymeric fabric may correspond to a random distribution of the therapeutic agent or drug.

In certain instances, the cover 324 may include at least a portion having variable porosity across the length or thickness of the cover 324. The fluid transport may be controlled by producing fibrils with higher or lower density, and/or with a substantially lesser or greater node count. In certain instances, the cover 324 may be controlled by producing fibrillated material with channels of progressively varying size, either diminishing or increasing. In certain instances, the narrowest channel size at the leading end of the distribution material opposes cellular ingress. In certain instances, the pore dimension of the cover 324 may be less than the size of nucleated cells (e.g., about 8 to about 20 microns), erythrocytes (e.g., about 8 microns), and platelets (e.g., about 2 microns).

The cover 324 being configured as a drug distribution material may facilitate diffusion of the therapeutic agent or drug into the intraperitoneal cavity, to an equilibrium. The rate of fluid exchange may be governed by concentration gradients and/or by the number of pores open for exchange (e.g., porosity of the distribution material). In certain instances, the structural features of the distribution material may offer an increased surface area resulting in increased permeability for diffusional fluid exchange. Porous material portions at the biological interface may be permeable to passive diffusion of solutes (therapeutic molecules). In certain instances, the architecture of the material (e.g., fibril and pore density) may be configured to exert a resistance to solution drainage at the exchange interface with host tissues.

In certain instances, restricted fluid motion due to boundary conditions may be influenced by the biological activity at the interface between the diffusion material and tissue. The cover 324 being configured as a drug distribution material may be configured to facilitate a bio-interface for unhindered therapeutic solute transport to the interstitial fluid and capillaries. In addition, the cover 324 may be configured to support host tissue anchoring, capillary growth, while minimizing foreign body encapsulation or chronic inflammation. In other instances the cover may be configured of a tight and porous materials or material composite that minimizes cellular ingress while allowing therapeutic outflow.

The cover 324 being configured as a drug distribution material may include free space and fenestrations within the diffusion material. The free space and fenestrations of the cover 324 may minimize dead space and optimize fluid transport. In certain instances, drug distribution material may be collapsible, a collapsible tube or include the catheters 608, 708 which may be collapsible. For example, the catheter 808 (or any of the catheters discussed herein) may include a self-collapsible elongate body 116 consisting of a self-collapsible composite material such as a porous ePTFE-elastomer (e.g., silicone or polyurethane) composite.

The self-collapsible elongate body 116, when a therapeutic agent is not infused therethrough, may be configured to collapse on itself. The self-collapsible elongate body 116 collapsing on itself seals the inner flow lumen 220 of the catheter and prevents or minimizes cell infiltration from the intraperitoneal space and foreign body reaction cascade within the lumen 200. As described in detail above, the proximal section 112 of the catheter 808 may be attached to an implantable pump for the delivery of the therapeutic agent. When the pump pressure is applied to deliver a therapeutic agent, the self-collapsible elongate body 116 expands (and may also elongate), thus opening the inner flow lumen 220 for the delivery of the therapeutic agent through the distal end 114. The expansion and/or elongation of the self-collapsible elongate body 116 under pump pressure can further enable detachment of any cells or organic deposits, which may have penetrated into the lumen, from the inner wall of the self-collapsible elongate body 116.

In certain instances, the cover 324 being configured as a drug distribution material may be configured to facilitate tissue anchoring and additionally prevent invasive cells from colonizing the material (e.g., macrophage fouling) or otherwise interfering with the release of the therapeutic agent. In certain instances and as described in detail above, the distribution material may be ePTFE configured to minimize or modulate fibrous capsule formation and establish a complete or partial barrier to biological tissues and cells. In certain instances, a portion of the partial barrier may also restrict permeation of body fluids such as blood, interstitial fluid, dissolved substances, or gases.

In addition, the inner wall of the elongate body 116 may include or be coated with a polymer layer, or may contain another polymer layer within the wall. In certain instances, the polymer layer may prevent a pH change of the drug or other therapeutic agent that is to be delivered. For example, the wall of inner flow lumen 220 of the catheter 808 may include a layer of or otherwise be coated with polyethylene, which serves as a barrier to permeation of carbon dioxide from the environment around the catheter, to maintain (or otherwise not influence) the pH balance of insulin delivered to the intraperitoneal space of a patient with diabetes. In other instances, other polymers or hydrophilic materials may be used corresponding to the therapeutic agent being used in treatment. The elongate body 116 and the wall of the inner flow lumen 220 (and/or outer surface) may further be coated with heparin, dexamethasone, or another bioactive agent to minimize fibrotic cell encapsulation around the catheter or a foreign body reaction inside the catheter 808.

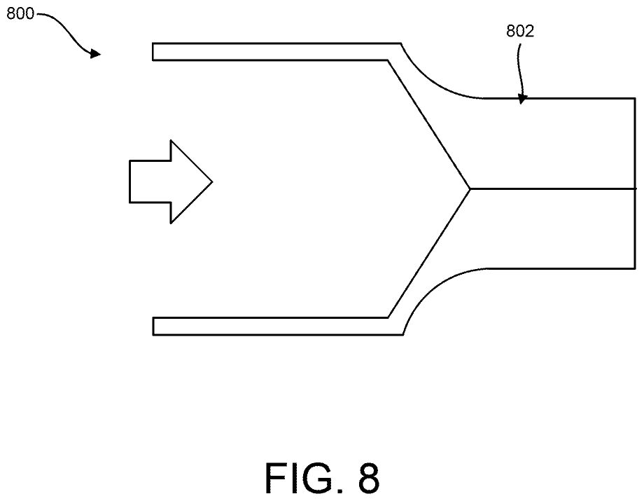

FIG. 8 is an example catheter tip 800, in accordance with various aspects of the present disclosure. The catheter tip 800 may be arranged at a distal end of an elongate body of any of the catheters discussed herein. A cover 222, as discussed in detail above, may at least partially cover the catheter tip 800.

The catheter tip 800, as shown, may be a pressure relief valve. In certain instances, the catheter tip 800 is configured as a self-closing tube section 802. The self-closing tube section 802 of the catheter tip 800 may open in response to pressure from a pump that forces a therapeutic agent through a catheter that the catheter tip 800 is coupled to. The self-closing tube section 802 of the catheter tip 800 may be formed of a porous ePTFE-elastomer (e.g., silicone or polyurethane) composite. In certain instances, the self-closing tube section 802 of the catheter tip 800 may be formed of an ePTFE reinforced silicone tube. For further discussion regarding example formation of the self-closing tube section 802, reference may be made to U.S. Pat. No. 9,849,629 by Zaggl, et al, which is incorporated by reference herein.

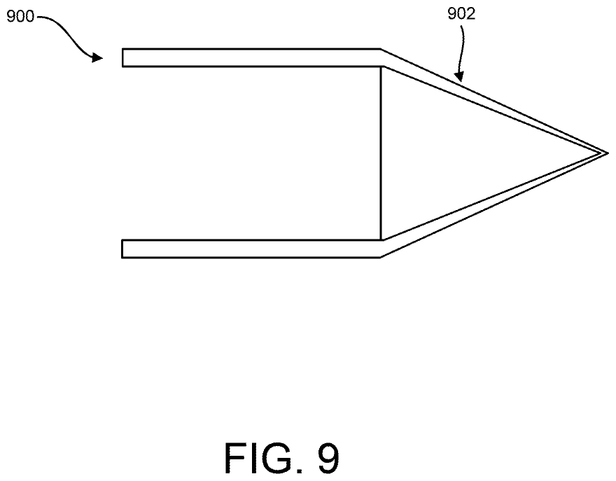

FIG. 9 is another example catheter tip 900, in accordance with various aspects of the present disclosure. The catheter tip 900 may be arranged at a distal end of an elongate body of any of the catheters discussed herein. A cover 222, as discussed in detail above, may at least partially cover the catheter tip 900.

In certain instances, the catheter tip 900 is configured as a duckbill section 902. The duckbill section 902 of the catheter tip 900 may open in response to pressure from a pump that forces a therapeutic agent through a catheter that the catheter tip 900 is coupled to. The pressure from the pump may open the duckbill section 902 and a release or absence of the pressure closes the duckbill section 902. Actuation or opening/closing of the duckbill section 902 may help release any foreign body response, inflammation, or cellular ingress deposits and maintain the opening substantially unobstructed for drug delivery through the catheter. The duckbill section 902 may be formed by pinching or forming an end of a tube with a liquid silicone injection into a mold. In other instances, the duckbill section 902 may be formed by an ePTFE/silicone composite tube on a mandrel.

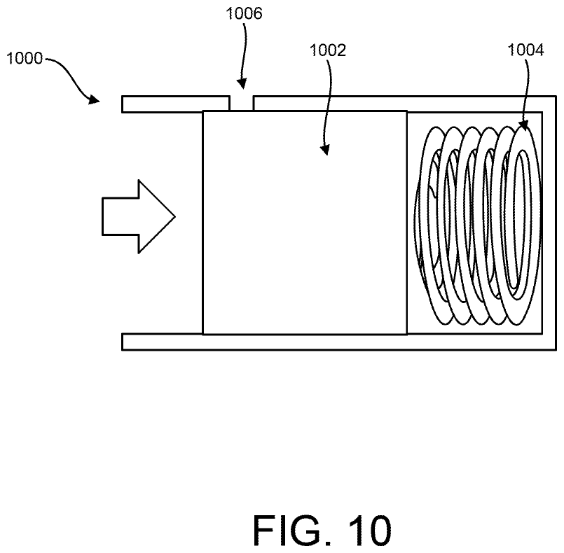

FIG. 10 is yet another example catheter tip 1000, in accordance with various aspects of the present disclosure. The catheter tip 1000 may be arranged at a distal end of an elongate body of any of the catheters discussed herein. A cover 222, as discussed in detail above, may at least partially cover the catheter tip 1000.

The catheter tip 1000, as shown, may be a pressure relief valve 1002. In certain instances, the pressure relief valve 1002 may be forced toward a distal end of the catheter tip 1000 and unblock an opening 1006 in the catheter tip 1000 in response to pressure from a pump that forces a therapeutic agent through a catheter that the catheter tip 1000 is coupled to. The pressure from the pump may force the pressure relief valve 1002 against a biasing mechanism 1004 (such as a spring or an elastomer) to allow the therapeutic agent to exit the biasing mechanism 1004 and release in the absence of the pressure to close off the opening 1006. Actuation or opening/closing of the pressure relief valve 1002 may help release any foreign body response, inflammation, or cellular ingress deposits and maintain the opening substantially unobstructed for drug delivery through the catheter.

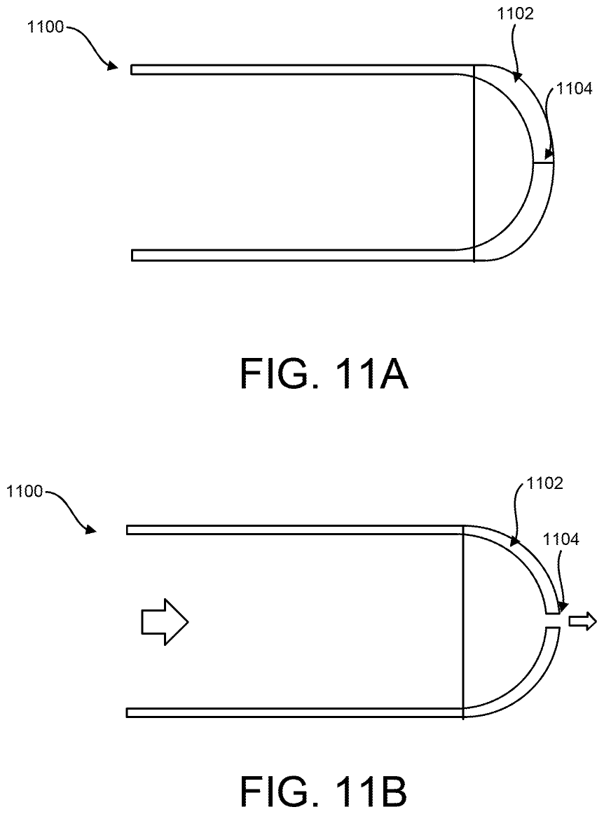

FIG. 11A is an example catheter tip 1100 in a first configuration, in accordance with various aspects of the present disclosure. The catheter tip 1100 may be arranged at a distal end of an elongate body of any of the catheters discussed herein. A cover 222, as discussed in detail above, may at least partially cover the catheter tip 1100.

The catheter tip 1100, as shown, may be a pressure distended elastomeric tip 1102. In certain instances, the pressure distended elastomeric tip 1102 may be forced toward a distal end of the catheter tip 1100 and open an opening 1104 in the catheter tip 1100 in response to pressure from a pump that forces a therapeutic agent through a catheter that the catheter tip 1100 is coupled to. The pressure from the pump may force open the opening 1104 (as shown in FIG. 11B) in the pressure distended elastomeric tip 1102 to allow the therapeutic agent to exit the opening 1104 and close the opening 1104 release in the absence of the pressure (as shown in FIG. 11A). The opening 1104 in the pressure distended elastomeric tip 1102 may be formed by stretching a sheet or piece of an elastomer or a porous ePTFE-elastomer (e.g., silicone or polyurethane) composite and piercing an opening in the material when stretched. In the absence of stretching, the sheet or piece of material and the opening shrink and are substantially closed.

In certain instances, during use, the pierced opening allows the therapeutic agent to exit the opening 1104 by bowing the pressure distended elastomeric tip 1102 outward. When the pressure of the pump or injection mechanism bowing the pressure distended elastomeric tip 1102 subsides, the pressure distended elastomeric tip 1102 will return to its original, compacted state and the pierced opening 1104 will be closed until the next distension. Actuation or opening/closing of the pressure distended elastomeric tip 1102 may help release any foreign body response, inflammation, or cellular ingress deposits and maintain the opening substantially unobstructed for drug delivery through the catheter.

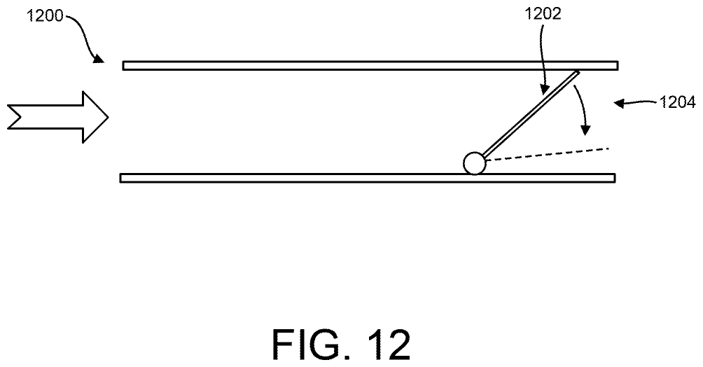

FIG. 12 is another example catheter tip 1200, in accordance with various aspects of the present disclosure. The catheter tip 1200 may be arranged at a distal end of an elongate body of any of the catheters discussed herein. A cover 222, as discussed in detail above, may at least partially cover the catheter tip 1200.

The catheter tip 1200, as shown, may be a valved structure. In certain instances, a valve 1202 arranged within the catheter tip 1200 may open in response to pressure from a pump that forces a therapeutic agent through a catheter that the catheter tip 1200 is coupled to. The pressure from the pump may open the valve 1202 such that the therapeutic agent may be released through a distal opening 1204, and a release or absence of the pressure closes the valve 1202. Actuation or opening/closing of the catheter tip 1200 may help release any foreign body response, inflammation, or cellular ingress deposits and maintain the opening substantially unobstructed for drug delivery through the catheter. The valve 1202 is confined within the catheter tip 1200 and is protected from the mechanical influence of the surrounding tissues that could otherwise oppose valve actuation. In certain instances, the catheter tip 1200 may include multiple valves 1202 that are each configured to open at the same or different prescribed pressures.

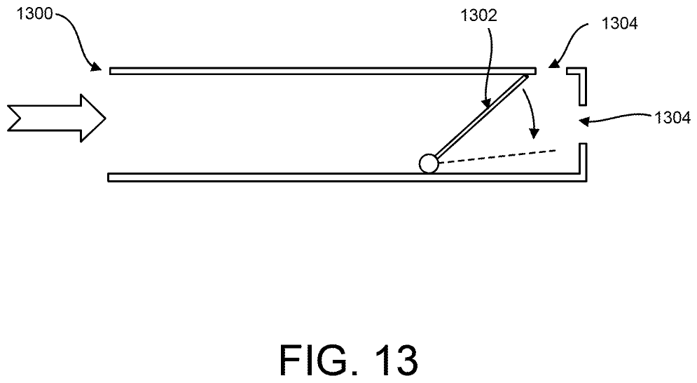

FIG. 13 is another example catheter tip 1300, in accordance with various aspects of the present disclosure. The catheter tip 1300 may be arranged at a distal end of an elongate body of any of the catheters discussed herein. A cover 222, as discussed in detail above, may at least partially cover the catheter tip 1300.

The catheter tip 1300, as shown, may be a valved structure. In certain instances, a valve 1302 arranged within the catheter tip 1300 may open in response to pressure from a pump that forces a therapeutic agent through a catheter that the catheter tip 1300 is coupled to. The pressure from the pump may open the valve 1302 such that the therapeutic agent may be released through at least one opening 1304 (side and/or distal opening in the catheter tip 1300), and a release or absence of the pressure closes the valve 1302. In certain instances, the catheter tip 1300 may include a side opening 1304 and a distal opening 1304. Actuation or opening/closing of the catheter tip 1300 may help release any foreign body response, inflammation, or cellular ingress deposits and maintain the opening substantially unobstructed for drug delivery through the catheter. The valve 1302 is confined within the catheter tip 1300 and is protected from the mechanical influence of the surrounding tissues that could otherwise oppose valve actuation. In certain instances, the catheter tip 1300 may include multiple valves 1302 that are each configured to open at the same or different prescribed pressures.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. An apparatus configured to be implanted within a patient, the apparatus comprising:
a catheter including a proximal section and a distal section configured to implant within the patient, an interior flow lumen, and at least one opening connected to the interior flow lumen for therapeutic agent delivery; and
a cover arranged about at least a portion of the distal section and configured to lessen at least one of a foreign body response, inflammation, and cellular ingress and maintain the opening substantially unobstructed for drug delivery through the catheter.

2. The apparatus of claim 1, wherein the cover is an ePTFE film.

3. The apparatus of claim 1, wherein the cover extends along the portion of the distal section between about 1 mm to about 100 mm from a distal end of the catheter.

4. The apparatus of claim 1, wherein the catheter is configured for drug delivery to an intraperitoneal space through the interior flow lumen and the cover includes a drug distribution material.

5. The apparatus of claim 4, wherein the catheter is an indwelling catheter configured to implant within the intraperitoneal space for up to 20 years.

6. The apparatus of claim 1, wherein the at least one opening is arranged at a distal end of the catheter.

7. The apparatus of claim 1, wherein the at least one opening includes a plurality of openings spaced about a circumference of the distal section of the catheter to enable uniform distribution of the therapeutic agent.

8. The apparatus of claim 7, wherein the cover is arranged over the plurality of openings.

9. The apparatus of claim 1, further comprising a sealed tip arranged at a distal end of the catheter.

10. The apparatus of claim 1, further comprising an interior layer arranged within the catheter along the interior flow lumen configured to lessen a foreign body response and inflammation.

11. The apparatus of claim 1, further comprising at least one of a bioactive agent or bioactive cover arranged on an exterior surface of the catheter.

12. The apparatus of claim 1, further comprising a self-closing tube section arranged at a distal end of an elongate body.

13. The apparatus of claim 12, wherein the self-closing tube section is configured to open in response to pressure from a pump that forces the therapeutic agent through the elongate body and close in response to the absence of the pressure.

14. The apparatus of claim 1, further comprising a catheter tip section arranged at a distal end of an elongate body that includes a valve configured to open in response to pressure from a pump that forces the therapeutic agent through the elongate body and close in response to the absence of the pressure.

15. The apparatus of claim 1, further comprising a pressure distended elastomeric tip arranged at a distal end of an elongate body that includes an opening configured to open in response to pressure from a pump that forces the therapeutic agent through the elongate body and close in response to the absence of the pressure.

16. A method of treatment, the method comprising the steps of:
providing a catheter including a proximal section, a distal section, an interior flow lumen, and at least one opening connected to the interior flow lumen for therapeutic agent delivery arranged at a distal end of the catheter;
inserting the distal end of the catheter into a patient; and
introducing the therapeutic agent to the interior flow lumen so that the therapeutic agent is delivered into the patient through the at least one opening.

17. The method of claim 16, wherein the catheter is further comprising a cover arranged about at least a portion of the distal section and configured to lessen at least one of a foreign body response, inflammation, and cellular ingress and maintain the opening substantially unobstructed for delivery of the therapeutic agent through the catheter.

18. The method of claim 17, wherein the cover is comprised of an ePTFE film.

19. The method of claim 17, wherein the cover extends along the portion of the distal section between about 1 mm to about 100 mm from the distal end of the catheter.

20. The method of claim 16, wherein the at least one opening includes a plurality of openings spaced about a circumference of the distal section of the catheter to enable uniform distribution of the therapeutic agent.

21. The method of claim 16, further comprising the step of controlling a flow of the therapeutic agent with a pump.

22. The method of claim 16, wherein the therapeutic agent comprises insulin.
